(19) United States
(12) Patent Application Publication
(10) Pub. No.: US 2004/0267032 A1
(43) Pub. Date: Dec. 30, 2004

(54) Process for recovering value-added products from pulping soap

(76) Inventors: Xiao Lu, Ste-Foy (CA); Christian Roy, Sillery (CA)

Correspondence Address: OGILVY RENAULT, 1981 MCGILL COLLEGE AVENUE, SUITE 1600, MONTREAL, QC, H3A2Y3, CA

(21) Appl. No.: 10/717,463

(22) Filed: Nov. 21, 2003

(30) Foreign Application Priority Data
Jun. 25, 2003 (CA) 2,433,246

(51) Int. Cl. C07J 9/00

(52) U.S. Cl. 552/540

(57) ABSTRACT

The invention relates to a process for isolating a phytosterol from a pulping soap. The process of the invention comprises the steps of: (a) treating the pulping soap with a first solvent selected from the group consisting of propan-2-one, methanol and mixtures thereof to remove water from the pulping soap and thereby obtain a liquid phase and a phytosterol-containing semi-solid phase; (b) separating the liquid phase and the semi-solid phase from one another; (c) extracting the phytosterol from the semi-solid phase with a second solvent selected from the group consisting of C₃-C₆ ketones, C₁-C₆ alkanols and mixtures thereof; and (d) recovering the phytosterol obtained in step (c). A process for isolating a mixture of salts of fatty acids and salts of resinic acids from a pulping soap is also disclosed.

### TECHNICAL FIELD

The present invention relates to improvements in the field of recovering value-added products from by-products of the pulp and paper industry. More particularly, the invention relates to a process for isolating a phytosterol from a pulping soap.

### BACKGROUND OF THE INVENTION

A few decades ago, the environment and pollution thereof became major concerns in our society. Many attempts have been made in various fields to reduce the emission of industrial waste material and toxic residues rejected in the environment. Recycling and reusing waste material became an efficient solution. Also, recovering value-added products from industrial waste material or from biomass is showing promising and encouraging results.

In the pulp and paper industry, since the '50s methods of extracting value-added chemicals such as phytosterols have been extensively used. In the Kraft process for preparing pulp from wood chips, a by-product called pulping soap or tall oil soap is generated. The expression “tall oil” is an anglicization of the Swedish “talloja”, meaning pine oil. The tall oil soaps generally have a limited commercial value and must be treated prior to extracting value-added products from them. Usually, a tall oil soap (or pulping soap) is first treated with sulfuric acid via an acidulation step to obtain a crude tall oil (CTO). Then, the latter is distillated to remove the volatile material such as distilled tall oil (DTO), leaving the tall oil pitch as a residue. Distilled tall oil is a mixture of resinic and fatty acids, and is used in lubricants and surfactants. The tall oil pitch is the by-product normally used for extracting and isolating phytosterols.

Extracting phytosterols from tall oil pitch is a process that has been frequently used but which has several serious drawbacks. The acidulation step in which the crude tall oil is obtained generates considerable quantities of another by-product, the so-called “brine” or “spent acid” which comprises about 15 w/w % of “salt cake” Na₂SO₄. Since the salt cake contains many impurities, it must be treated and purified to be of commercial interest. Also, the tall oil pitch is a very complex material comprising a plurality of organic compounds. Isolating phytosterols from the latter is a complicated task since it is difficult to obtain high purity phytosterols in good yields. In fact, the tall oil pitch includes impurities such as long-chain alcohols and acids which are particularly difficult to separate from the phytosterols since they all have similar high molecular weights. U.S. Pat. Nos. 2,573,891, 2,715,638, 2,835,682 and 3,840,570 all disclose processes for isolating sterols from tall oil pitch and these processes all suffer from the above-mentioned disadvantages.

U.S. Pat. No. 5,770,749 discloses a process for isolating phytosterols from a pulping soap. In a first step, the pulping soap is added to a solvent mixture comprising water and ketone and the resulting mixture is then continuously extracted with a hydrocarbon such as hexane. The extraction product is dried over sodium sulfate (Na₂SO₄) and evaporated to obtain a creamy precipitate which is eventually purified by crystallization. This process has shown improvements with respect to the above-mentioned processes using tall oil pitch, but still has several drawbacks. In fact, the process disclosed in U.S. Pat. No. 5,770,749 generates a solvent mixture comprising water, a ketone and a hydrocarbon, which cannot be easily recycled in view of its complexity. The latter process uses large quantities of sodium sulfate as a drying agent and thus also generates considerable amounts of solid residues, which must eventually be treated. The extraction step of this process is very time-consuming since 24 hours are required to perform the extraction. The process allows one to isolate only phytosterols from the pulping soap. Since the phytosterols recovered represent only about 3 weight % of the total quantity of pulping soap treated and, considering the fact that this process generates considerable amounts of solid residues such as contaminated hydrated sodium sulfate, the process has a very low recovery yield of waste material. When comparing the quantity of the value-added products obtained and the quantity of waste products generated at the end of the process, it is clear that this process has a low efficacy in terms of valorization of waste material and minimization of waste material generated and production costs.

### SUMMARY OF THE INVENTION

It is therefore an object of the present invention to avoid the above drawbacks and to provide a process for recovering value-added products from a pulping soap, which is not time-consuming and does not generate considerable quantities of waste material.

According to a first aspect of the invention, there is provided a process for isolating a phytosterol from a pulping soap, comprising the steps of:

a) treating the pulping soap with a first solvent selected from the group consisting of propan-2-one, methanol and mixtures thereof to remove water from the pulping soap and thereby obtain a liquid phase and a phytosterol-containing semi-solid phase;

b) separating the liquid phase and the semi-solid phase from one another;

c) extracting the phytosterol from the semi-solid phase with a second solvent selected from the group consisting of C₃-C₆ ketones, C₁-C₆ alkanols and mixtures thereof; and

d) recovering the phytosterol obtained in step (c).

According to a second aspect of the invention, there is provided a process for isolating a mixture of salts of fatty acids and salts of resinic acids from a pulping soap, comprising the steps of:

a) treating the pulping soap with a first solvent selected from the group consisting of propan-2-one, methanol and mixtures thereof to remove water from the pulping soap and thereby obtain a first liquid phase and semi-solid phase;

b) separating the first liquid phase and the semi-solid phase from one another;

c) treating the semi-solid material with a second solvent selected from the group consisting of C₃-C₆ ketones, C₁-C₆ alkanols and mixtures thereof to obtain a second liquid phase and a solid material comprising the mixture of salts of fatty acids and salts of resinic acids;

d) separating the second liquid phase and the solid material from one another; and

e) recovering the solid material.

Applicant has found quite surprisingly that by using the process according to the first aspect of the invention, it was possible to isolate a phytosterol from a pulping soap by utilizing a simple solvent so as to minimize the quantity of waste material generated. Moreover, such a process is not time-consuming.

Applicant has also found quite surprisingly that by using the process according to the second aspect of the invention, it was possible to isolate from the pulping soap a mixture of salts of resinic and fatty acids, the latter mixture being useful as a mineral flotation collector for the froth flotation of minerals.

### DESCRIPTION OF PREFERRED EMBODIMENTS

In the process according to the first aspect of the invention, step (c) is preferably carried out by:

i) treating the semi-solid phase with the second solvent to obtain a solution of the phytosterol in the second solvent, and a solid material; and

ii) separating the solution and the solid material from one another, preferably, by a filtration or a decantation.

Step (d) of the process according to the first aspect of the invention can be carried out by evaporating the second solvent. The second solvent is preferably condensed and recycled to step (c).

The process according to the first aspect of the invention can further include the step of recovering the solid material. Preferably, the solid material is recovered prior to recovering the phytosterol or both are recovered simultaneously. Such a solid material comprises a mixture of salts of fatty acids and salts of resinic acids.

In the process according to the second aspect of the invention, step (d) is preferably carried out by filtration or decantation.

The process according to the second aspect of the invention can further comprises the step of recovering the second liquid phase, the second liquid phase comprising the second solvent and a phytosterol. The phytosterol is preferably isolated by evaporating the second solvent. The second solvent can be condensed and recycled to step (c).

The process according to the second aspect of the invention can also comprise the step of acidifying the salts to obtain a mixture of the corresponding fatty acids and resinic acids.

As already indicated, the mixture of salts of fatty acids and salts of resinic acids can be used as a mineral flotation collector for the froth flotation of minerals. The main principles of flotation is based on the creation of the difference in physicochemical surface properties among various mineral particles. These surface properties can be described as hydrophobic or hydrophilic in terms of flotation. The mineral particles in hydrophobic state will be able to attach to the air-bubbles whereas the particles in hydrophilic state will not attach to the air-bubbles.

In the flotation process, air-bubbles are introduced into the pulp treated by chemicals in the flotation cells. The objective mineral particles then attach to the bubbles and usually are transferred to the froth to form mineralized froth (concentrate). At same time, the other mineral particles still stay in the pulp or tailing. Then, the separation between the different minerals is achieved.

To achieve the satisfactory separation conditions, it is necessary to use a plurality of chemicals such as flotation reagents. Those chemicals are called respectively as collector, frother, regulator according to their function in the flotation process. Mineral collectors are used to increase the surface hydrophobicity of the objective mineral particles since minerals naturally have a low hydrophobicity due to their surface polar characteristics. Minerals are usually classified in five groups according to their polarity. Correspondingly, different types of surfactants known as collector are used to the pulp and agitation is necessary to allow them adsorption onto the mineral surface during the conditioning stage.

Mineral collectors can be non-ionising (liquid and non-polar hydrocarbons) or ionizing (anionic or cationic). Among the anionic collectors, long hydrocarbon chains bearing carboxylic acids, sulphates, sulphonates, xanthates or dithiophosphates may be cited. The ionizing collectors are more commonly used since they are heteropolar i.e. a non-polar hydrocarbon group and a polar group as previously mentioned. As example, salts of carboxylic and more particularly acids fatty acids, are used for the flotation of calcium, barium, strontium, magnesium, carbonates of non-ferrous metals, soluble salts of alkali metals and alkaline earth metals. Also, xanthates and dithiophosphates are very powerful and selective for the flotation of sulphide minerals. Usually, the interaction between the collector and the mineral is in such that the collector adsorbs on the mineral particles with their non-polar ends towards the bulk pulp and therefore imparts particle hydrophobicity. Many advantages can be obtained from an economic and environment-friendly mineral collector. In particular, such a collector obtained from waste material would be of an important interest.

In the process according to the first and second aspects of the invention, the pulping soap used can comprise from 25 to 50% and preferably from 27 to 30% by weight of water, based on the total weight of the pulping soap.

The first solvent used in the process according to the first and second aspects of the invention is preferably methanol and more preferably propan-2-one.

Step (a) of the process according to the first and second aspects of the invention can be carried out at a temperature ranging from 5 to 30° C., preferably from 20 to 25° C. In step (a), water contained in the pulping soap is preferably transferred into the liquid phase so that the semi-solid phase obtained in step (b) can be substantially free of Water. The expression “substantially free of water” as used herein in respect of the semi-solid phase obtained in step (b) refers to a residual water content of less than about 25 weight %. The residual water content of the semi-solid phase obtained in step (b) is generally less than 20%, preferably less than 15%, more preferably less than 10% and even more preferably less than 5% by weight, based on the total weight of the semi-solid phase. The liquid phase obtained in step (b) can be treated to separate the first solvent and the water from one another and the first solvent can thus recycled to step (a).

Step (c) of the process according to the first and second aspects of the invention can be carried out at a temperature ranging from 20 to 55° C., preferably from 40 to 50° C.

The weight ratio first solvent/pulping soap used in the process according to the first and second aspects of the invention can range from 0.3 to 1.5, preferably from 0.5 and 1.0. The weight ratio second solvent/pulping soap can range from 4 to 20, preferably from 5 to 10. The first solvent and the second solvent can be the same.

In the process according to the first and second aspects of the invention, the second solvent is preferably a C₃-C₆ ketone and more preferably a C₃-C₆ ketone selected from the group consisting of propan-2-one, butan-2-one, pentan-2-one, pentan-3-one and mixtures thereof. Propan-2-one is particularly preferred.

The process according to the first aspect of the invention can further comprise the step of purifying the recovered phytosterol. The phytosterol is preferably purified by filtration, crystallization or a combination thereof. Crystallization is particularly preferred. The recovered phytosterol preferably comprises a mixture of β-sitosterol, campesterol, and stigmasterol. The mixture can comprises from 50 to 80% and preferably from 65 to 75% by weight of β-sitosterol, from 10 to 30% and preferably from 15 to 25% by weight of campesterol, and from 5 to 20% and preferably from 17 to 20% by weight of stigmasterol, based on the total weight of the mixture. More preferably, the mixture comprises about 69% by weight of β-sitosterol, 19% by weight of campesterol and about 12% by weight of stigmasterol, based on the total weight of the mixture.

The fatty acids obtained in the process according to the first and second aspects of the invention are preferably C₆-C₂₆ carboxylic acids such as nonanoic acid, lauric acid, palmitic acid, heptadecanoic acid, stearic acid, oleic acid, octadec-8-enoic acid, octadec-9-enoic acid, octadeca-9,11-dienoic acid, octadeca-9,12-dienoic acid, nonadecanoic acid, nonadec-10-enoic acid, eicosanoic acid, docosanoic acid,. tetracosanoic acid, pentacosanoic acid, hexacosanoic acid, etc. The resinic acids are preferably selected from the group consisting of abietic acid, palustric acid and mixtures thereof.

The salts obtained in the process according to the first and second aspects of the invention can comprise metal salts in which the metal is preferably selected from the group consisting of Al, Ca, Fe, K, Mg, Na and mixtures thereof. More preferably, the metal is sodium. The solid material can comprise from 25 to 75% and preferably from 45 to 65% by weight of sodium salts of fatty acids, based on the total weight of the mixture. The solid material can also comprise from 25 to 75% and preferably from 35 to 55% by weight of sodium salts of resinic acids, based on the total weight of the mixture.

The phytosterol obtained in the process according to the first and second aspects of the invention can be used in cosmetic compositions, in pharmaceutical compositions or as active agent in functional food. The phytosterol can also be used to reduce blood cholesterol level or to reduce the risks of prostate disorders, preferably benign prostatic hyperplasia or BPH. In addition, the phytosterol can be used as a precursor in the synthesis of stanols, steroids, hydroxycortisones or spiranolactones.

The salts obtained in the process according to the first or second aspect of the invention can be used as a mineral flotation collector or as an additive in paints, inks, adhesives or surfactants.

The process according to the first and second aspects of the invention can be performed in less than 5 hours. In particular, the step of extracting the phytosterol from the semi-solid phase with the second solvent according to the first aspect of the invention can be achieved in less than 3 hours. Steps (c) and (d) according to the second aspect of the invention can be performed in less than 3 hours.

### BRIEF DESCRIPTION OF THE DRAWINGS

The following non-limiting examples illustrate the invention, reference being made to the accompanying drawings in which:

FIG. 1 is a plot comparing a mineral flotation collector of the following invention and lauric acid during a hematite flotation and recovery experiment;

FIG. 2 is a plot illustrating the flotation rate of the mineral flotation collector of FIG. 1 during a hematite flotation and recovery experiment;

FIG. 3 is a plot illustrating the flotation rate of lauric acid during a hematite flotation and recovery experiment;

FIG. 4 is a plot illustrating the effect of pH on the mineral flotation collector of FIG. 1 during a hematite flotation and recovery experiment; and

FIG. 5 is a graph comparing the mineral flotation collector of FIG. 1 and lauric acid during a quartz flotation and recovery experiment.

### EXAMPLE I

A quantity of 100 g of a pulping soap was mixed in a beaker with 60 g of propan-2-one at room temperature to obtain a liquid phase comprising water and propan-2-one, and a semi-solid phase comprising the value-added products to be isolated. The liquid phase was then separated from the semi-solid phase by means of a decantation. The liquid phase was recovered so as to recycle the propan-2-one.

Then, in order to extract phytosterols from said semi-solid phase, the latter was treated with propan-2-one at room temperature (9×100 g) so as to obtain a mixture including a solution of the phytosterols in propan-2-one, and a solid material comprising a mixture of salts of fatty acids and salts of resinic acids. The solution was separated from the solid material by filtrating the mixture. 50 g of the mixture of salts of fatty acids and salts of resinic acids were thus obtained. The solution was concentrated until a volume of 50 mL was obtained and then the phytosterols were crystallized. The crystallized phytosterols were then washed with water and dried to obtain 3 g of phytosterols as white needles. The phytosterols isolated were β-sitosterol, campesterol and stigmasterol in a 69:19:12 ratio.

### EXAMPLE 2

A quantity of 100 g of a pulping soap was mixed in a beaker with 60 g of propan-2-one at room temperature to obtain a liquid phase comprising water and propan-2-one, and a semi-solid phase comprising the value-added products to be isolated. The liquid phase was then separated from the semi-solid phase by means of a decantation. The liquid phase was recovered so as to recycle the propan-2-one.

Then, in order to extract phytosterols from said semi-solid phase, the latter was treated with propan-2-one at 45° C. (5×100 g) so as to obtain a mixture including a solution of the phytosterols in propan-2-one, and a solid material comprising a mixture of salts of fatty acids and salts of resinic acids. The solution was separated form the solid material by filtrating the mixture. 48 g of the mixture of salts of fatty acids and salts of resinic acids were thus obtained. The solution was concentrated until a volume of 50 mL was obtained and then, the phytosterols were crystallized. The crystallized phytosterols were then washed with water and dried to obtain 3 g of phytosterols as white needles. The phytosterols isolated were β-sitosterol, campesterol and stigmasterol in a 69:19:12 ratio.

### EXAMPLE 3

The mixture of salts of fatty acids and salts of resinic acids obtained in example 1 was tested for its use as a mineral flotation collector. For the flotation test, a Denver flotation cell with volume of 1.5 L was used. In the experiments, the minerals used were hematite and quartz having a fine particle size (100% less than 100 mesh). A sample of the mixture of salts of fatty acids and salts of resinic acids as obtained in example 1, (hereinafter referred to as “XL-SSRS”) and lauric acid were tested for comparison purposes. Since these two mineral collectors were found to have a frothing function, no frothing agent was needed.

For each experiment, hematite flotation and quartz flotation, a pulp was first prepared in the cell by mixing 700 grams of a mineral (hematite or quartz) with 2100 ml of tap water. The pulp was then agitated for 5 minutes at 1500 rpm. The pH of the pulp was controlled by NaOH or HCl. Air was introduced into the cell at a flow rate of 34.5 mL/min. During the flotation test, samples of froth (concentrates) were collected at predetermined periods of time. These samples were filtered and dried for analysis.

To evaluate the results of the flotation experiments, two indexes are usually used: flotation recovery and concentrate grade. The concentrate grade is the mineral or element content in the concentrate. The flotation recovery is calculated with the mineral or element content in the concentrate divided by the mineral or element content in the feed. A high concentrate grade with a high flotation recovery means a perfect flotation result.

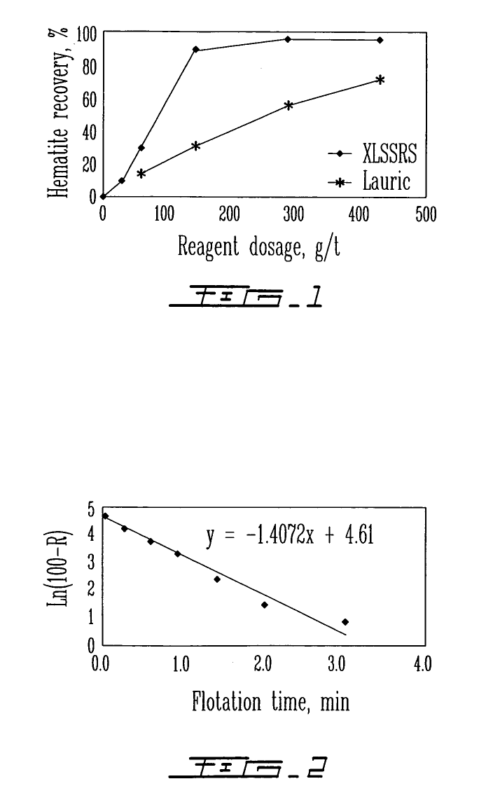

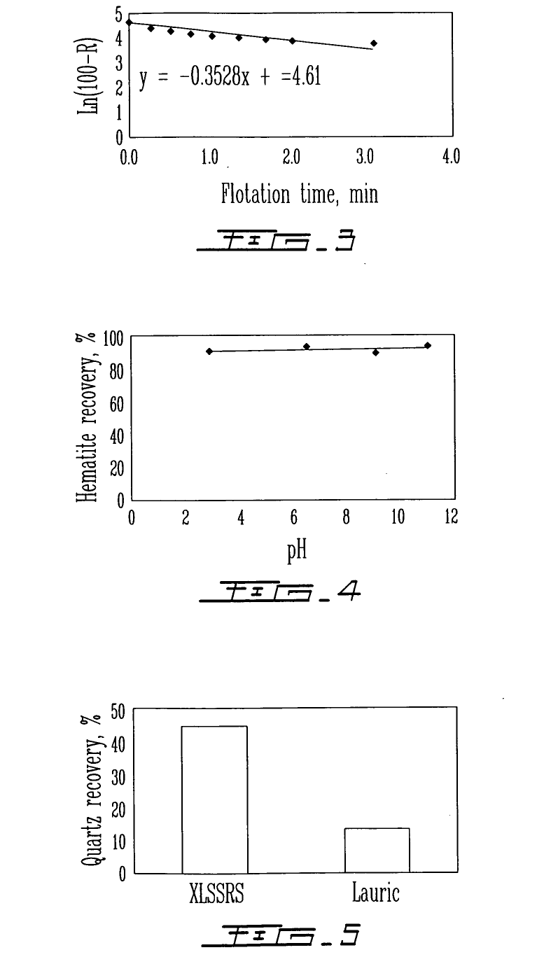

From FIG. 1, it can be seen that the flotation recovery of hematite increases rapidly when the dosage of XL-SSRS increases from 0 to 150 g/t feed. After 300 g/t, the recovery can be as high as 97%. FIG. 1 demonstrates that XL-SSRS is a strong hematite collector. It also demonstrates that XL-SSRS is more efficient than lauric acid for recovering hematite. In particular, XL-SSRS provided a hematite recovery which is 24% higher than lauric acid. FIGS. 2 and 3 demonstrate that XL-SSRS has a flotation rate which is faster than lauric acid (1.41 min′ vs. 0.35 min⁻¹).

The pH effect on the action of the mineral collector is also an important factor that must be considered. FIG. 4 clearly demonstrates that XL-SSRS is not very sensitive to the variation of the pulp pH value in the range of 3 to 11. In fact, its efficacy for recovering hematite is not affected by such variations.

Comparative quartz flotation experiments were also conducted using XL-SSRS and lauric acid. The results obtained are shown in FIG. 5. It can clearly be seen that XL-SSRS provided higher quartz recovery than lauric acid. XL-SSRS must therefore be considered as a stronger quartz collector than lauric acid.

XL-SSRS was acidified and tested via a gas chromatography analysis in order to determine its composition. The results are as follows:

| Component | % by weight |
|---|---|
| Fatty acids: | |
| tridecanoic acid | 0.02 |
| tetradecanoic acid | 0.44 |
| dodecadienoic acid | 0.12 |
| pentadecanoic acid | 0.03 |
| 14-methylpentadecanoic acid | 0.10 |
| hexadecenoic acid | 0.05 |
| headecenoic acid | 0.04 |
| hexadecenoic acid | 0.01 |
| hexadecanoic acid | 0.93 |
| 14-methylhexadecanoic acid | 1.00 |
| heptadecanoic acid | 0.09 |
| 6,9,12-octadecatrienoic acid | 2.52 |
| 6,9,12-octadecatrienoic acid | 2.52 |
| 9,12-octadecadienoic acid | 2.42 |
| 9,12-octadecadienoic acid | 5.42 |
| oleic acid (9-octadecenoic acid) | 6.18 |
| linolenic acid (9,12,15-octadecatrienoic acid) | 2.61 |
| 10-octadecadienoic acid | 1.70 |
| 7,10-octadecadienoic acid | 2.71 |
| linoleic acid (9,12-octadecadienoic acid) | 0.93 |
| 7,10-octadecadienoic acid | 1.38 |
| nanodecandienoic acid | 0.31 |
| nanodecenoic acid | 0.44 |
| 7,10-octadecadienoic acid | 0.33 |
| nanodecanoic acid | 0.03 |
| nanodecatrienoic acid | 0.57 |
| 6,9,12-octadecatrienoic acid | 1.35 |
| arachidic acid (eicosanoic acid) | 4.08 |
| heneicosanoic acid | 0.13 |
| tricosanoic acid | 0.19 |
| eicosanebioic acid | 0.13 |
| lignoceric acid (tetracosanoic acid) | 1.06 |
| pentacosanoic acid | 0.02 |
| pentacosanoic acid | 0.21 |
| docosanedioic acid | 0.18 |
| hexacosanoic acid | 0.13 |
| heptacosanoic acid | 0.03 |
| 5,8,11-eicosatrienoic acid | 0.72 |
| pentacosanoic acid | 0.21 |
| docosanedioic acid | 0.18 |
| hexacosanoic acid | 0.13 |
| heptacosanoic acid | 0.26 |
| Other fatty acids | 12.12 |
| Resinic acids: | |
| decahydro-1,4-dimethyl-7-isopropyl-1- | 0.67 |
| phenanthrene carboxylic acid | |
| dodecahydro-7-ethenyl-1,4,7-trimethyl-1- | 0.34 |
| phenanthrene carboxaldehyde | |
| dodecahydro-1,4,7-trimethyl-7-ethenyl-1- | 3.02 |
| phenanthrene carboxylic acid | |
| dodecahydro-1,4,7-trimethyl-7-ethenyl-1- | 2.84 |
| phenanthrene carboxylic acid | |
| dodecahydro-1,4,7-trimethyl-7-ethenyl-1- | 3.07 |
| phenanthrene carboxylic acid | |
| dodecahydrotrimethyl-7-ethenyl-1- | 1.83 |
| phenanthrene carboxylic acid | |
| dodecahydro-1,4,7-trimethyl-1-phenanthrene | 0.21 |
| carboxylic acid | |
| dodecahydro-1,4,7-trimethyl-7-ethenyl-1- | 4.49 |
| phenanthrene carboxylic acid | |
| dodecahydro-1,4,7-trimethyl-7-ethenyl-1- | 6.30 |
| phenanthrene carboxylic acid | |
| octahydro-1,4-dimethyl-7-isopropyl-1- | 0.10 |
| phenanthrene carboxylic acid | |
| dodecahydro-7-ethenyl-1,4,7-trimethyl-1- | 0.21 |
| phenanthrene carboxylic acid | |
| Abietic acid | 10.10 |
| octahydro-1,4-dimethyl-7-isopropenyl-1- | 0.17 |
| phenanthrene carboxylic acid | |
| dodecahydro-1,4-dimethyl-7-isoproplydene- | 4.30 |
| 1-phenanthrene carboxylic acid | |
| dodecahydro-1,4,7-trimethyl-1-phenanthrene | 0.15 |
| carboxylic acid | |
| dodecahydro-7-ethenyl-1,4,7-trimethyl-1- | 0.46 |
| phenanthrene carboxylic acid | |
| decahydro-1,4-dimethyl-7-isopropenyl-1- | 1.21 |
| phenanthrene carboxylic acid | |
| decahydro-1,4-dimethyl-7-isopropenyl-1- | 0.98 |
| phenanthrene carboxylic acid | |
| Other resinic acids | 1.50 |
| Others: | |
| non-identified compounds during the GC | 4.02 |
| analysis | |
| TOTAL | 100.00 |

It should be understood from the above-mentioned examples that the ratio of β-sitosterol/campesterol/stigmasterol in the isolated phytosterols can vary depending on the composition of the pulping soap treated. Analogously, the proportion by weight of salts of fatty acids and salts of resinic acids can also vary depending on the composition of the pulping soap. The composition of the latter will vary depending on the type of wood used in the pulp and paper industry.

We claim:

1. A process for isolating a phytosterol from a pulping soap, comprising the steps of:
a) treating said pulping soap with a first solvent selected from the group consisting of propan-2-one, methanol and mixtures thereof to remove water from said pulping soap and thereby obtain a liquid phase and a phytosterol-containing semi-solid phase;
b) separating the liquid phase and the semi-solid phase from one another;
c) extracting said phytosterol from said semi-solid phase with a second solvent selected from the group consisting of C₃-C₆ ketones, C₁-C₆ alkanols and mixtures thereof; and
d) recovering said phytosterol.

2. The process of claim 1, wherein said first solvent is propan-2-one.

3. The process of claim 1, wherein said first solvent is methanol.

4. The process of claim 1, wherein in step (a), water contained in the pulping soap is transferred into the liquid phase so that the semi-solid phase obtained in step (b) is substantially free of water.

5. The process of claim 1, wherein the liquid phase obtained in step (b) is treated to separate said first solvent and said water from one another and wherein the first solvent thereby separated is recycled to step (a).

6. The process of claim 1, wherein said second solvent is propan-2-one.

7. The process of claim 1, wherein step (c) is carried out at a temperature ranging from 20 to 55° C.

8. The process of claim 1, wherein in step (d) said second solvent is recovered and recycled to step (c).

9. The process of claim 1, further including the step of recovering said solid material.

10. The process of claim 9, wherein said solid material comprises a mixture of salts of fatty acids and salts of resinic acids.

11. The process of claim 1, further including the step of purifying the phytosterol recovered in step (d).

12. The process of claim 1, wherein the recovered phytosterol comprises a mixture of β-sitosterol, campesterol and stigmasterol.

13. The process of claim 12, wherein said mixture comprises from 50 to 80% by weight of β-sitosterol, from 10 to 30% by weight of campesterol and from 5 to 20% by weight of stigmasterol, based on the total weight of the mixture.

14. The process of claim 13, wherein said mixture comprises about 69% by weight of β-sitosterol, 19% by weight of campesterol and about 12% by weight of stigmasterol, based on the total weight of the mixture.

15. A process for isolating a phytosterol from a pulping soap, comprising the steps of:
a) treating said pulping soap with a first solvent selected from the group consisting of propan-2-one, methanol and mixture thereof to remove water from said pulping soap and to obtain a liquid phase and a phytosterol-containing semi-solid phase;
b) separating the liquid phase and the semi-solid phase from one another;
c) treating said semi-solid material with a second solvent consisting of propan-2-one to obtain a solution of said phytosterol in said second solvent, and a solid material;
d) separating said solution and said solid material from one another;
e) recovering the phytosterol from said solution; and
f) recovering said solid material.

16. The process of claim 15, wherein in step (a), water contained in the pulping soap is transferred into the liquid phase so that the semi-solid phase obtained in step (b) is substantially free of water.

17. The process of claim 15, wherein the liquid phase obtained in step (b) is treated to separate said first solvent and said water from one another and wherein the first solvent thereby separated is recycled to step (a).

18. The process of claim 15, wherein step (d) is carried out by filtration or decantation.

19. The process of claim 15, wherein step (e) is carried out by evaporating said second solvent.

20. The process of claim 19, wherein said second solvent is condensed and recycled to step (c).

21. The process of claim 15, wherein step (f) is performed prior to step (e) or simultaneously therewith.

22. The process of claim 15, further including the step of purifying the phytosterol recovered in step (e).

23. The process of claim 15, wherein the recovered phytosterol comprises a mixture of β-sitosterol, campesterol and stigmasterol.

24. The process of claim 23, wherein said mixture comprises from 50 to 80% by weight of β-sitosterol, from 10 to 30% by weight of campesterol and from 5 to 20% by weight of stigmasterol, based on the total weight of the mixture.

25. The process of claim 24, wherein said mixture comprises about 69% by weight of β-sitosterol, 19% by weight of campesterol and about 12% by weight of stigmasterol, based on the total weight of the mixture.

26. The process of claim 15, wherein said solid material comprises a mixture of salts of fatty acids and salts of resinic acids.

27. The process of claim 26, wherein said salts comprise metal salts in which the metal is selected from the group consisting of Al, Ca, Fe, K, Mg, Na and mixtures thereof.

28. The process of claim 27, wherein said salts are sodium salts.

29. A process for isolating a mixture of salts of fatty acids and salts of resinic acids from a pulping soap, comprising the steps of:
a) treating said pulping soap with a first solvent selected from the group consisting of propan-2-one, methanol and mixtures thereof to remove water from said pulping soap and thereby obtain a first liquid phase and semi-solid phase;
b) separating the first liquid phase and the semi-solid phase from one another;
c) treating said semi-solid material with a second solvent selected from the group consisting of C₃-C₆ ketones, C₁-C₆ alkanols and mixtures thereof to obtain a second liquid phase and a solid material comprising said mixture of salts of fatty acids and salts of resinic acids;
d) separating said second liquid phase and said solid material from one another; and
e) recovering said solid material.

30. The process of claim 29, wherein in step (a), water contained in the pulping soap is transferred into the liquid phase so that the semi-solid phase obtained in step (b) is substantially free of water.

31. The process of claim 29, wherein the liquid phase obtained in step (b) is treated to separate said first solvent and said water from one another and wherein the first solvent thereby separated is recycled to step (a).

32. The process of claim 29, wherein said second solvent is propan-2-one.

33. The process of claim 29, further comprising the step of recovering the second liquid phase, said second-liquid phase comprising said second solvent and a phytosterol.

34. The process of claim 33, wherein said phytosterol is isolated by evaporating said second solvent.

35. The process of claim 29, wherein said second solvent is condensed and recycled to step (c).

36. The process of claim 29, further including the step of acidifying said salts to obtain a mixture of the corresponding fatty acids and resinic acids.

37. The process of claim 29, wherein said solid material comprises from 25 to 75% by weight of sodium salts of fatty acids, based on the total weight of the mixture.

38. The process of claim 29, wherein said solid material comprises from 25 to 75% by weight of sodium salts of resinic acids, based on the total weight of the mixture.

39. The process of claim 29, wherein said salts comprise metal salts in which the metal is selected from the group consisting of Al, Ca, Fe, K, Mg, Na and mixtures thereof.

40. The process of claim 39, wherein said salts are sodium salts.
